(19) United States
(12) Patent Application Publication
(10) Pub. No.: US 2010/0197910 A1
(43) Pub. Date: Aug. 5, 2010

(54) PROCESS FOR PREPARATION OF PRULIFLOXACIN USING NOVEL INTERMEDIATES

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrapradesh (IN)

(21) Appl. No.: 11/916,649

(22) PCT Filed: Nov. 17, 2006
(86) PCT No.: PCT/IN06/00458

§ 371 Date: Dec. 5, 2007

(51) Int. Cl. C07D 513/04; C07F 5/02

(52) U.S. Cl. 544/229; 544/361

(57) ABSTRACT

The present invention provides a novel process for the preparation of the prulifloxacin intermediate, 6-fluoro-1-methyl-4-oxo-7-(1-piperazinyl)-4H-[1,3] thiazeto[3,2-a]quinoline-3-carboxylic acid, thereby producing prulifloxacin and its pharmaceutical acceptable acid addition salts thereof in high purity and in high yield using novel intermediates in lesser reaction time. Thus, for example, ethyl 6,7-difluoro-1-methyl-4-oxo-4H-[1,3]thiazeto[3,2-a]quinoline-3-carboxylate is reacted with boric acid in the presence of acetic anhydride and acetic acid to give a borane compound, which is then condensed with piperazine in the presence of acetonitrile and dimethylsulfoxide, followed by treatment with potassium hydroxide solution to give 6-fluoro-1-methyl-4-oxo-7-(1-piperazinyl)-4H-[1,3] thiazeto [3,2-a]quinoline-3-carboxylic acid.

### FIELD OF THE INVENTION

The present invention provides a novel and commercially viable process for prulifloxacin intermediate, 6-fluoro-1-methyl-4-oxo-7-(1-piperazinyl)-4H-[1,3]thiazeto[3,2-a]quinoline-3-carboxylic acid, thereby producing prulifloxacin and its pharmaceutical acceptable acid addition salts thereof in high purity and in high yield using novel intermediates in lesser reaction time.

### BACKGROUND OF THE INVENTION

European Patent No. 315828 disclosed a variety of quinoline carboxylic acid derivatives and pharmaceutically acceptable salts thereof. These compounds are exhibiting antibacterial activity and useful as remedies for various infectious diseases. Among them prulifloxacin, chemically (±)-6-Fluoro-1-methyl-7-[4-(5-methyl-2-oxo-1,3-dioxolen-4-ylmethyl)-1-piperazinyl]-4-oxo-4H-[1,3]thiazeto[3,2-a]quinoline-3-carboxylic acid is a fluoroquinolone antibacterial prodrug which shows potent and broad-spectrum antibacterial activity both in vitro and in vivo. Prulifloxacin also showed superior activity against strains of Enterobacteriaceae and Pseudomonas aeruginosa. Prulifloxacin is represented by the following structure:

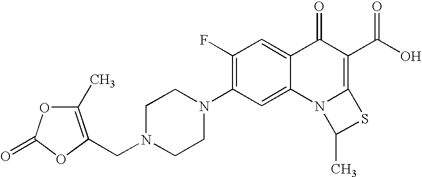

Processes for the preparation of prulifloxacin and related compounds were disclosed in European Patent No. 315828 and UK Patent Application No. GB 2190376.

In the preparation of prulifloxacin, 6-fluoro-1-methyl-4-oxo-7-(1-piperazinyl)-4H-[1,3]thiazeto[3,2-a]quinoline-3-carboxylic acid of formula I:

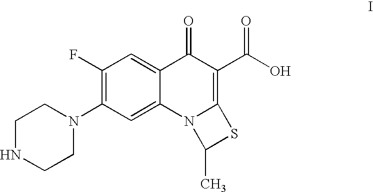

is a key intermediate. According to the UK Patent Application No. GB 2190376, the compound of the formula I was prepared by the reaction of 3,4-difluroaniline with carbon disulfide and triethylamine to give triethylammonium N-(3,4-difluorophenyl)dithio carbamate, which by reaction with ethyl chloroformate and triethylamine in chloroform is converted into 3,4-difluorophenyl isothiocyanate, followed by reaction with diethyl malonate and KOH in dioxane affords the potassium salt, which is then treated with methoxymethyl chloride in dimethylformamide to give diethyl 1-(3,4-difluorophenylamino)-1-(methoxymethylthio)-methylene-malonate. The cyclization of the thio compound at 240° C. in diphenyl ether affords ethyl 6,7-difluoro-4-hydroxy-2-methoxymethylthioquinoline-3-carboxylate, which by treatment with HCl in ethanol gives ethyl 6,7-difluoro-4-hydroxy-2-mercaptoquinoline-3-carboxylate. The cyclization of the mercapto compound with 1,1-dibromoethane by means of potassium carbonate and potassium iodide in hot dimethylformamide yields ethyl 6,7-difluoro-1-methyl-4-oxo-4H-[1,3]thiazeto[3,2-a]quinoline-3-carboxylate, which is condensed with piperazine in dimethylformamide to afford ethyl 6-fluoro-1-methyl-4-oxo-7-(1-piperazinyl)-4H-[1,3]thiazeto[3,2-a]quinoline-3-carboxylate, which is then subjected to hydrolysis with potassium hydroxide in hot tert-butanol to give the compound of formula I.

The compound of formula I obtained by the process described in the UK Patent Application No. GB 2190376 is not satisfactory from purity point of view, the reaction between ethyl 6,7-difluoro-1-methyl-4-oxo-4H-[1,3]thiazeto[3,2-a]quinoline-3-carboxylate and piperazine requires longer time about 48 hours to complete, the yield obtained is not satisfactory, and the process also involves column chromatographic purifications. Methods involving column chromatographic purifications cannot be used for large-scale operations, thereby making the process commercially not viable.

According to the European Patent No. 315828, prulifloxacin is prepared by reacting 6-fluoro-1-methyl-4-oxo-7-(1-piperazinyl)-4H-[1,3]thiazeto[3,2-a]quinoline-3-carboxylic acid with 4-bromomethyl-5-methyl-1,3-dioxolen-2-one in presence of potassium bicarbonate in dimethylformamide.

However, a need still remains for an improved and commercially viable process of preparing pure prulifloxacin that will solve the aforesaid problems associated with process described in the prior art and will be suitable for large-scale preparation, in terms of simplicity, purity and yield of the product.

One object of the present invention is to provide a novel process for preparation of prulifloxacin intermediate.

Another object of the present invention is to provide a process for preparing prulifloxacin and its pharmaceutical acceptable acid addition salts thereof in high purity and in high yield using novel intermediates in lesser reaction time.

### DETAILED DESCRIPTION OF THE INVENTION

In accordance with the present invention, there is provided a novel process for preparing 6-fluoro-1-methyl-4-oxo-7-(1-piperazinyl)-4H-[1,3]thiazeto [3,2-a]quinoline-3-carboxylic acid of formula I:

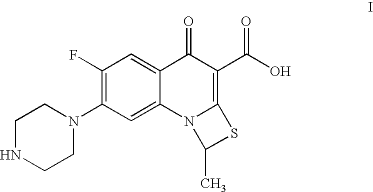

which comprises:
a) reacting the difluoro-quinoline compound of formula II:

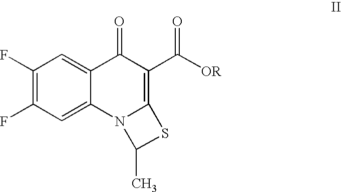

wherein R represents hydrogen atom or alkyl containing 1 to 4 carbon atoms;
with boric acid of formula III:

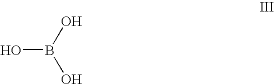

in presence of acetic anhydride and acetic acid to give borane compound of formula IV:

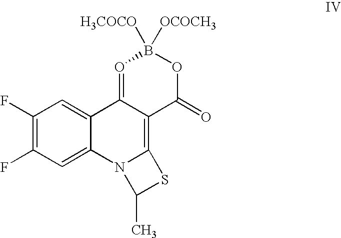

b) reacting the borane compound of formula IV with piperazine of formula V:

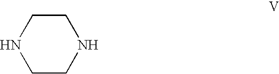

to give piperazine compound of formula VI:

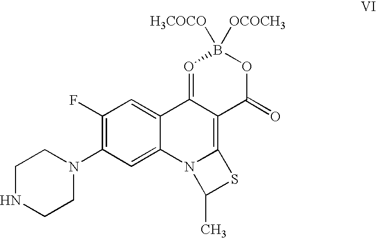

c) treating the compound of formula VI with an alkaline metal hydroxide, carbonate or bicarbonate to obtain the compound of formula I.

Prulifloxacin and pharmaceutically acceptable acid addition salts of prulifloxacin can be prepared by using the compound of formula I by known methods for example as described in the European Patent No. 315828.

Borane compound of the formula IV and VI are novel and forms part of the invention.

Preferably the reaction in step (a) is carried out at about 30° C. to reflux temperature more preferably at about 80° C. to reflux temperature and still more preferably at reflux temperature.

The compounds of formula II, wherein R is hydrogen, methyl or ethyl are preferable. The compounds of formula II, wherein R is hydrogen or ethyl are more preferable.

Preferably, the borane compound of formula IV formed is isolated as solid by conventional means.

Preferably the reaction in step (b) is carried out at about 30-100° C., more preferably at about 50-95° C. and still more preferably at about 70-90° C.

Preferably the reaction in step (b) is carried out in a solvent selected from hydrocarbon solvents such as n-hexane, n-heptane and cyclohexane; chlorinated hydrocarbon solvents such as methylene chloride, ethylene chloride and chloroform; acetonitrile, tetrahydrofuran, 1,4-dioxane and a mixture thereof, and more preferable solvent is acetonitrile.

The compound of formula V in step (b) may be used as free base or as an acid addition salt form. If the compound of formula V is used as an acid addition salt, it is preferred to convert the salt to the free base before reacting with the compound of formula IV.

Preferable alkaline metal hydroxide used in step(c) is sodium hydroxide or potassium hydroxide; preferable alkaline metal carbonate is sodium carbonate or potassium carbonate; and preferable alkaline metal bicarbonate is sodium bicarbonate or potassium bicarbonate. More preferable alkaline metal hydroxide is aqueous sodium hydroxide.

The reaction mass containing the compound of formula I obtained in step(c) may be subjected to usual work up. The reaction mass may be used directly in the next step to produce finally prulifloxacin or its pharmaceutically acceptable acid addition salts, or the compound of formula I may be isolated and used in the next step.

The compound of formula II is known and can be obtained from known procedures.

The invention will now be further described by the following examples, which are illustrative rather than limiting.

### Examples

### Example 1

### Step-I:

Acetic anhydride (24 ml) and acetic acid (11 ml) are added to boric acid (3.5 gm) under stirring at 25-30° C., the contents are heated to reflux and then stirred for 3 hours at reflux. The reaction mass is cooled to 100° C., ethyl 6,7-difluoro-1-methyl-4-oxo-4H-[1,3]thiazeto[3,2-a]quinoline-3-carboxylate (20 gm) is added at 100° C., the contents are heated to reflux and then refluxed for 2 hours. The reaction mass is cooled to 25-35° C., toluene (200 ml) is added under stirring, the reaction mass is cooled to 5° C. and then stirred for 1 hour at 5-10° C. Filtered the solid, washed with 20 ml of toluene and then dried to give 25.5 gm of 6,7-difluoro-1-methyl-4-oxo-4H-[1,3]thiazeto[3,2-a]quinoline-3-carboxylate-O³,O⁴/bis/acetato-O/-borone.

### Step-II:

Acetonitrile (125 ml), dimethylsulfoxide (125 ml) and piperazine (13.8 gm) are added to 6,7-difluoro-1-methyl-4-oxo-4H-[1,3]thiazeto[3,2-a]quinoline-3-carboxylate-O³,O⁴/bis/acetato-O/-borone (25.5 gm, obtained in step-I) under stirring at 25-35° C., the contents are heated to 85° C. and then stirred for 3 hours at 80-85° C. to form a clear solution. The solution is cooled to 10° C. and then stirred for 1 hour at 10-15° C. Filtered the solid, washed with 25 ml of acetonitrile and then dried to give 26 gm of 6-fluoro-1-methyl-4-oxo-7-(1-piperazinyl)-4H-[1,3]thiazeto[3,2-a]quinoline-3-carboxylate-O³,O⁴/bis/acetato-O/-borone.

### Step-III:

Water (155 ml), potassium hydroxide (17 gm) are added to 6-fluoro-1-methyl-4-oxo-7-(1-piperazinyl)-4H-[1,3]thiazeto[3,2-a]quinoline-3-carboxylate-O³,O⁴/bis/acetato-O/-borone (26 gm, obtained in step-II) under stirring at 25-35° C., the contents are heated to 65° C. and then stirred for 4 hours at 60-65° C. The reaction mass is cooled to 25° C., filtered the undesired solid on hi-flow bed and then pH of the resulting filtrate is adjusted to 7-7.5 with 50% HCl solution at 25-30° C. The separated solid is stirred for 1 hour at 25-30° C., filtered the solid, washed with 35 ml of water and then dried to give 17 gm of 6-fluoro-1-methyl-4-oxo-7-(1-piperazinyl)-4H-[1,3]thiazeto [3,2-a]quinoline-3-carboxylic acid (HPLC Purity: 98.5%).

### Example 2

### Step-I:

Acetic anhydride (12 ml) and acetic acid (5.5 ml) are added to boric acid (1.25 gm) under stirring at 25-30° C., the contents are heated to reflux and then stirred for 3 hours at reflux. The reaction mass is cooled to 100° C., 6,7-difluoro-1-methyl-4-oxo-4H-[1,3]thiazeto[3,2-a]quinoline-3-carboxylic acid (10 gm) is added at 100° C., the contents are heated to reflux and then refluxed for 3 hours. The reaction mass is cooled to 50° C., toluene (100 ml) is added under stirring at 50° C., the resulting mass is cooled to 10° C. and then stirred for 1 hour at 10-15° C. Filtered the solid, washed with 20 ml of toluene and then dried to give 10 gm of 6,7-difluoro-1-methyl-4-oxo-4H-[1,3]thiazeto[3,2-a]quinoline-3-carboxylate-O³,O⁴/bis/acetato-O/-borone.

### Step-II:

Acetonitrile (50 ml), dimethylsulfoxide (50 ml) and piperazine (5.5 gm) are added to 6,7-difluoro-1-methyl-4-oxo-4H-[1,3]thiazeto[3,2-a]quinoline-3-carboxylate-O³,O⁴/bis/acetato-O/-borone (10 gm, obtained in step-I) under stirring at 25-35° C., the contents are heated to 85° C. and then stirred for 3 hours at 80-85° C. to form a clear solution. The solution is cooled to 10° C. and then stirred for 1 hour at 10-15° C. Filtered the solid, washed with 10 ml of acetonitrile and then dried to give 10.4 gm of 6-fluoro-1-methyl-4-oxo-7-(1-piperazinyl)-4H-[1,3]thiazeto[3,2-a]quinoline-3-carboxylate-O³,O⁴/bis/acetato-O/-borone.

### Step-III:

Water (62 ml), potassium hydroxide (7 gm) are added to 6-fluoro-1-methyl-4-oxo-7-(1-piperazinyl)-4H-[1,3]thiazeto[3,2-a]quinoline-3-carboxylate-O³,O⁴/bis/acetato-O/-borone (10.4 gm, obtained in step-II) under stirring at 25-35° C., the contents are heated to 65° C. and then stirred for 4 hours at 60-65° C. The reaction mass is cooled to 25° C., filtered the undesired solid on hi-flow bed and then pH of the resulting filtrate is adjusted to 7-7.5 with 50% HCl solution at 25-30° C. The separated solid is stirred for 30 minutes at 25-30° C., filtered the solid, washed with 20 ml of water and then dried to give 68 gm of 6-fluoro-1-methyl-4-oxo-7-(1-piperazinyl)-4H-[1,3]thiazeto [3,2-a]quinoline-3-carboxylic acid (HPLC Purity: 98.6%).

### Example 3

Acetonitrile (560 ml) and potassium bicarbonate (8 gm) are added to 6-fluoro-1-methyl-4-oxo-7-(1-piperazinyl)-4H-[1,3]thiazeto[3,2-a]quinoline-3-carboxylic acid (14 gm, obtained as per the processes described in examples 1 and 2) under stirring at 25-30° C., the contents are cooled to 15° C. and then the solution of 4-bromomethyl-5-methyl-1,3-dioxolen-2-one (10 gm) in acetonitrile (140 ml) is added at 15-20° C. for 30 to 45 minutes. The contents are stirred for 25 hours at 25 to 30° C., filtered and the resulting filtrate is distilled under vacuum. To the residue added acetonitrile (70 ml), cooled the mass to 20° C. and then stirred for 1 hour to 1 hour 30 minutes at 20-25° C. Filtered the solid, washed the solid with 15 ml of chilled acetonitrile and then dried to give 16 gm of prulifloxacin crude (HPLC Purity: 98.8%).

To the prulifloxacin crude (obtained above) added acetonitrile (200 ml) at 25-30° C., the contents are heated to reflux and then refluxed for 30 minutes. To the reaction mass added activated carbon (5 gm) and refluxed for 15 minutes. The reaction mass is filtered on hi-flo bed, the resulting filtrate is cooled to 20° C. and then stirred for 3-4 hours at 20-25° C. Filtered the solid, washed with 20 ml of acetonitrile and then dried to give 14 gm of prulifloxacin (HPLC Purity: 99.9%).

1. A process for the preparation of 6-fluoro-1-methyl-4-oxo-7-(1-piperazinyl)-4H-[1,3]thiazeto [3,2-a]quinoline-3-carboxylic acid of the formula I: 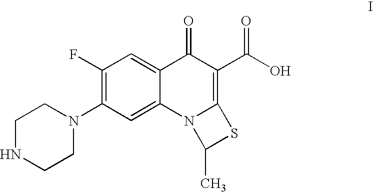
which comprises:
a) reacting a difluoro-quinoline compound of the formula II: 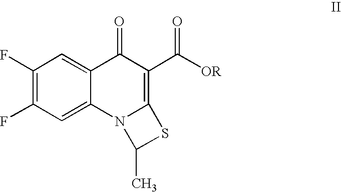
wherein R represents a hydrogen atom or an alkyl group containing 1 to 4 carbon atoms;
with boric acid of the formula III: 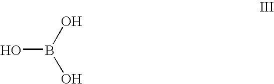
in the presence of acetic anhydride and acetic acid to give a borane compound of the formula IV: 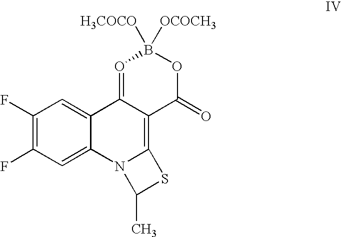
b) reacting the borane compound of the formula IV with a piperazine of the formula V: 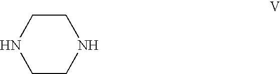
to give a piperazine compound of the formula VI: 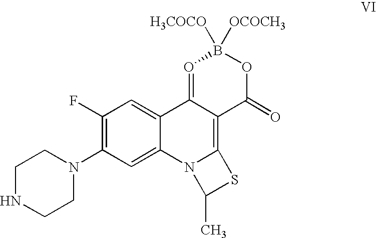
c) treating the compound of the formula VI with an alkaline metal hydroxide, carbonate or bicarbonate to obtain the compound of the formula I.

2. The process as claimed in claim 1, wherein the reaction in step (a) is carried out at about 30° C. to reflux temperature.

3. The process as claimed in claim 2, wherein the reaction is carried out at about 80° C. to reflux temperature.

4. The process as claimed in claim 3, wherein the reaction is carried out at reflux temperature.

5. The process as claimed in claim 1, wherein the reaction in step (b) is carried out in a solvent selected from hydrocarbon solvents, chlorinated hydrocarbon solvents, acetonitrile, tetrahydrofuran, 1,4-dioxane and a mixture thereof.

6. The process as claimed in claim 5, wherein the hydrocarbon solvent is n-hexane, cyclohexane or n-heptane.

7. The process as claimed in claim 5, wherein the chlorinated hydrocarbon solvent is methylene chloride.

8. The process as claimed in claim 5, wherein the solvent is acetonitrile.

9. The process as claimed in claim 1, wherein the reaction in step (b) is carried out at about 30-100° C.

10. The process as claimed in claim 9, wherein the reaction in step (b) is carried out at about 50-95° C.

11. The process as claimed in claim 10, wherein the reaction in step (b) is carried out at about 70-90° C.

12. The process as claimed in claim 1, wherein the alkaline metal hydroxide is sodium hydroxide or potassium hydroxide, alkaline metal carbonate is sodium carbonate or potassium carbonate, and alkaline metal bicarbonate is sodium bicarbonate or potassium bicarbonate.

13. The process as claimed in claim 12, wherein the alkaline metal hydroxide is sodium hydroxide.

14. Compound of formula IV:

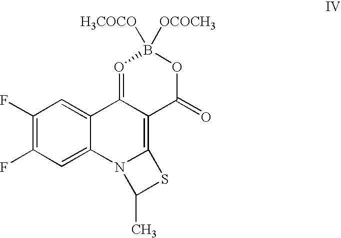

15. Compound of formula VI: [chemical structure]
